(19) United States
(12) Patent Application Publication
(10) Pub. No.: US 2008/0068773 A1
(43) Pub. Date: Mar. 20, 2008

(54) PERSONAL BODY GROUNDING SYSTEMS TO REDUCE AND PREVENT INFLAMMATION

(75) Inventors: A. Clinton Ober, West Covina, CA (US); James L. Oschman, Dover, NH (US)

Correspondence Address: KELLY LOWRY & KELLEY, LLP, 6320 CANOGA AVENUE, SUITE 1650, WOODLAND HILLS, CA, 91367, US

(73) Assignee: EARTH FX, INC., Palm Springs, CA (US)

(21) Appl. No.: 11/852,905

(22) Filed: Sep. 10, 2007

Related U.S. Application Data
(60) Provisional application No. 60/825,614, filed on Sep. 14, 2006.

(51) Int. Cl. A61N 1/14; H05F 3/02

(52) U.S. Cl. 361/224; 361/220

(57) ABSTRACT

The invention discloses methods and systems for conductively coupling the body and more specifically the bare feet of an animal or human body to the earth in situations where conductive contact with the earth is otherwise impossible, such as inside a home or other building or in a bed. Various embodiments of the invention allow earth's mobile surface charge of free electrons to flow from the earth to the body, (similar to that which occurs naturally when an animal or human is standing barefoot on the earth), for the purpose of neutralizing excess or residual immune system-produced reactive oxygen species free radicals that cause physiological deficiencies and disturbances in the body such as acute or chronic inflammation.

### BACKGROUND OF THE INVENTION

The present invention generally relates to devices and methods for reducing and preventing chronic inflammation. More particularly, the present invention relates to personal grounding devices and systems that inhibit the expression of chronic inflammation in an animal or human. This inhibition is achieved by conductively coupling the body with the earth to conduct earth's mobile surface charge of free electrons from the earth to the body. This coupling restores the body's natural supply of free electrons to reduce and prevent residual or collateral immune responses produced by reactive oxygen species (ROS) free radicals, which oxidize tissue. When an animal or human body is naturally charged with earth's free electrons, residual immune system-produced free radicals have a readily available source of free electrons to rapidly reduce their oxidative state. This inhibits free radical oxidation of healthy tissue and thereby inhibits the promotion and manifestation of chronic inflammation and inflammation-related health disorders in the body. Free electrons from the earth do not interfere with the normal and vital immune responses to tissue damage and/or infection and subsequent tissue repair processes; instead, electrons have a natural protective effect on healthy or undamaged cells and tissues near a site of trauma. In other words, free electrons from the earth augment and focus the body's natural responses to injury.

It is well established, though not widely known, that the surface of the earth possesses a limitless and continuously renewed supply of free or mobile electrons. The earth's surface is electrically conductive and is maintained at a negative potential by the global atmospheric electrical circuit. The universal conductivity of the earth varies somewhat from place to place, depending upon water and mineral content, vegetation and other factors. However, these factors have relatively little effect on the ability of an earth connection to allow free electrons to flow from the earth to the body or vice versa. Further, any conductive object, coupled with the earth, will conduct earth's mobile charge of free electrons and equalize with it and thereafter maintain the negative potential of the earth. Human and animal bodies are conductive and when they are coupled with the earth they also conduct and become saturated with the earth's negative surface charge of mobile free electrons. Humans and animals and their respective progenitors lived in conductive contact with the earth, i.e., barefoot, during their primary evolutionary period. The immune system's reactive oxygen species immune response mechanisms also developed during the period when humans and animals lived in a natural grounded state. The inventor has linked loss of natural grounding via the integration of plastic and other insulative materials in our living environments as a contributor to the rapid rise in inflammation-related health disorders. Non-conductive natural and synthetic polymer-based soled footwear, floor coverings, bedding and the like now insulate most humans and domestic animals from routine conductive contact with the earth. Clinical case studies show that when the body is conductively coupled with the earth, chronic inflammation and related health disorders resolve naturally.

The primary defense mechanism of the body is the immune systems release of reactive oxygen specie (ROS) free radicals. The immune response is triggered by injury or disease. White blood cells are constantly circulating within the tissues, essentially poised to respond to the presence of viruses, bacteria or injured cells (Garrood T L, Lee L, and Pitzalis C, 2006; Molecular mechanisms of cell recruitment to inflammatory sites: general and tissue-specific pathways; Rheumatology 45(3):250-260). When an injury occurs, chemical, electrical and other messages are produced that attract white blood cells to the injured or diseased tissue. Chemical signals from the injured tissue can attract other, more specialized cells (Springer T A, 1995; Traffic signals on endothelium for lymphocyte recirculation and leukocyte emigration; Annual Reviews of Physiology 57:827-872).

Part of the inflammatory response involves various immune cells, known as neutrophils, as well as other types of phagocytes, which secrete an abundance of powerful oxidizing agents (free radicals) in a process known as the respiratory burst. The respiratory burst consists of a complex mix of very reactive molecules such as hydrogen peroxide, oxidized halogens, chloramines and oxidizing radicals such as hydroxyl radical, —OH, that aid in the destruction of invading microorganisms. To restore their electrical neutrality, these agents tear electrons from the structures of invading organisms and damaged cells, rapidly destroying them.

While ROS free radicals are obviously vital to the immune response, problems arise when the process does not completely wind down after an injury or site of disease has been cleared of pathogens and cellular debris. Under these conditions, residual free radicals begin to oxidize healthy tissues. Oxidation of healthy tissue by the ROS free radicals then leads to the release of additional chemical signals that re-stimulate the immune system. The immune system responds by delivering more free radicals, establishing a destructive or vicious cycle that can continue indefinitely. Some biomedical researchers refer to this as silent inflammation, and it is being recognized as the culprit behind almost every modern chronic disease.

Scientists have known for a long time that the inflammatory response can backfire, causing a host of autoimmune diseases. There are about 80 such disorders, the most common being rheumatoid arthritis, multiple sclerosis, Hashimoto's thyroiditis, Graves' disease, Lupus, and Crohn's disease. The idea that chronic inflammation could be involved in other diseases began to gain credence when doctors realized that stomach ulcers were not caused by stress or spicy food, but by inflammation triggered during bacterial infection (Marshall B J and Warren J R, 1984. Unidentified curved bacilli in the stomach of patients with gastritis and peptic ulceration; Lancet 1(8390):1311-1315. Also see the 2005 Nobel Prize for Physiology or Medicine awarded jointly to Barry J. Marshall and J. Robin Warren for their discovery of “the bacterium Helicobacter pylori and its role in gastritis and peptic ulcer disease.”)

It has also long been known that Type 1 diabetes is linked to inflammation—the body's immune system attacks the cells that make insulin. New research is suggesting that Type 2 diabetes, the kind that generally occurs in adulthood, often begins with insulin resistance, in which cells stop responding properly to insulin. Doctors now know that during chronic inflammation, one of the chemicals released is tumor necrosis factor (TNF) which makes cells more resistant to insulin. The TNF connection also helps explain why obesity, particularly abdominal obesity, leads to diabetes. Fat cells used to be thought of as storage depots for energy, as metabolically inactive; now we know that fat cells are little hotbeds of inflammation—excess fat in the belly is a great source of inflammation.

Recently evidence has accumulated to show that inflammation is a major factor in far more conditions than autoimmune diseases, ulcers and diabetes. Some of the most thorough documentation of the role of inflammation in disease has come from the research of Dr. P M Ridker and his colleagues at the Center for Cardiovascular Disease Prevention, and Division of Cardiology, Brigham and Women's Hospital and Harvard Medical School in Boston, Mass., USA (Ridker P M Hennekens C H, Buring J E, and Rifai N, 2000; C-reactive protein and other markers of inflammation in the prediction of cardiovascular disease in women; New England Journal of Medicine 342(12):836-43). Suspecting that inflammation is involved in the pathogenesis of cardiovascular events, these researchers measured the levels of markers of inflammation in a prospective controlled study among 28,263 apparently healthy postmenopausal women over a mean follow-up period of three years. They assessed the risk of cardiovascular events associated with a variety of established inflammatory markers, including high-sensitivity C-reactive protein (hs-CRP), homocysteine and a variety of lipid (e.g. cholesterol) and lipoprotein measurements. Cardiovascular events were defined as death from coronary heart disease, nonfatal myocardial infarction or stroke, or the need for coronary revascularization procedures. Of 12 markers measured, hs-CRP proved to be the strongest predictor of the risk of cardiovascular events. Markers of inflammation, when combined with lipid measurements, were significantly better at predicting risk than models based on lipid levels alone (P<0.001). The levels of hs-CRP and serum amyloid A were significant predictors of risk even in the subgroup of women with normal cholesterol levels. The study concluded that adding the measurement of the inflammatory marker, C-reactive protein, to screening based on lipid levels could improve the identification of persons at risk for cardiovascular events. In 2004, a group in Taipei, Taiwan essentially confirmed these results in a study of non-diabetic patients (Leu H B, Lin C P, Lin W T, Wu T C and Chen J W, 2004; Risk stratification and prognostic implication of plasma biomarkers in nondiabetic patients with stable coronary artery disease: the role of high-sensitivity C-reactive protein; Chest. 126(4):1032-9).

In 2001, Ridker and colleagues studied the risk factors for systemic atherosclerosis in 14,916 initially healthy US male physicians. Again, total cholesterol-HDL-C ratio and CRP were the strongest independent predictors of development of peripheral arterial disease. C-reactive protein provided additive prognostic information over standard lipid measures (Ridker P M, Stampfer M J, and Rifai N, 2001. Novel risk factors for systemic atherosclerosis: a comparison of C-reactive protein, fibrinogen, homocysteine, lipoprotein(a), and standard cholesterol screening as predictors of peripheral arterial disease; JAMA 285(19):2481-2485).

In 2001, another group at Massachusetts General Hospital and Harvard Medical School, Boston, Mass. USA reported on high levels of CRP associated with hypopituitarism and growth hormone deficiency. This phenomenon had already been reported in men, and this study extended the findings to women. Hypopituitary women have increased levels of IL-6 and CRP, both of which are inflammatory markers of atherosclerosis (Sesmilo G, Miller K K, Hayden D, and Klibanski A, 2001; Inflammatory cardiovascular risk markers in women with hypopituitarism; J Clin Endocrinol Metab. 86(12):5774-5781).

In 2002, Ridker and colleagues reported measurements of C-reactive protein and LDL cholesterol in 27,939 apparently healthy American women who were then followed for a mean of eight years for the occurrence of myocardial infarction, ischemic stroke, coronary revascularization, or death from cardiovascular causes. They found that base-line levels of each marker had a strong linear relation with the incidence of cardiovascular events (Ridker P M Rifai N, Rose L, Buring J E, and Cook N R; 2002; Comparison of C-reactive protein and low-density lipoprotein cholesterol levels in the prediction of first cardiovascular events; New England Journal of Medicine, 347(20):1557-65).

Further study by Ridker and colleagues revealed a correlation between chronic inflammation and sudden cardiac death (Alenghat F J, and Ingber D E, 2002; Mechanotransduction: All Signals Point to Cytoskeleton, Matrix, and Integrins; Science's STKE: http://stke.sciencemag.org/cgi/content/full/OC_sigtrans;2002/119/pe6).

As a result of these studies, and others like them, the American Heart Association and the Centers for Disease Control and Prevention recommended in 2003 that doctors include a test for free radicals in their medical check-ups, to determine a patient's risk for heart disease (Pearson T A Mensah G A Alexander R W, et al., 2003; Markers of Inflammation and Cardiovascular Disease Application to Clinical and Public Health Practice. A Statement for Healthcare Professionals From the Centers for Disease Control and Prevention and the American Heart Association; Circulation, 107:499-511). Subsequently there has been a veritable explosion of research into the association of inflammation and inflammatory markers with a wide range of chronic illnesses. Today, nearly every branch of medicine and surgery includes the study of inflammation (Alenghat F J, supra.)

Inflammation is now thought to be the underlying mechanism of more than 80 chronic illnesses, in addition to the autoimmune disorders mentioned above. These chronic illnesses involve almost every human organ system. They include diseases of the nervous, gastrointestinal, endocrine and respiratory systems as well as the skin and connective tissues. In all of these diseases, the underlying problem is similar—the body's immune system is attacking the very organs it was designed to protect. And inflammation in one organ can be associated with problems in other organs.

For example, in 2004, Knight and colleagues studied the association among kidney function, inflammatory biomarker levels, and coronary events. A total of 244 women with no history of cardiovascular disease that subsequently had incident coronary events were matched to 486 control subjects. High-sensitivity CRP (hs-CRP), IL-6, and sTNFR I and II levels were all significantly associated with an increased odds of coronary events in women with reduced kidney function but not in women with normal kidney function. Kidney dysfunction is associated with increased odds of coronary events, and inflammation, as assessed by higher inflammatory biomarker levels, specifically hs-CRP, IL-6, and soluble tumor necrosis factor receptor I and II were significantly associated with coronary events only in women with reduced kidney function (Knight E L Rimm E B Pai J K, Rexrode K M, Cannuscio C C, Manson J E, Stampfer M J, and Curhan G C, 2004; Kidney dysfunction, inflammation, and coronary events: a prospective study; J Am Soc Nephrol, 15(7):1897-903).

Subsequent ongoing research has confirmed a role for inflammation in atherosclerosis (Folsom A R, Chambless L E, Ballantyne C M, Coresh J, Heiss G, Wu K K, Boerwinkle E, Mosley T H Jr, Sorlie P, Diao G, and Sharrett A R, 2006; An assessment of incremental coronary risk prediction using C-reactive protein and other novel risk markers: the atherosclerosis risk in communities study; Arch Intern Med. 166(13):1368-73), diabetes (Ben-Mahmud B M, Chan W H, Abdulahad R M, Datti A, Orlacchio A, Kohner E M, and Chibber R, 2006; Clinical validation of a link between TNF-alpha and the glycosylation enzyme core 2 GlcNAc-T and the relationship of this link to diabetic retinopathy; Diabetologia, 49(9):2185-2191), rheumatoid arthritis (Datta D, Ferrell W R, Sturrock R D, Jadhav S T, and Sattar N, 2007; Inflammatory suppression rapidly attenuates microvascular dysfunction in rheumatoid arthritis; Atherosclerosis 192(2):391-195), multiple sclerosis (Pleasure D, Soulika A, Singh S K, Gallo V, and Bannerman P, 2006; Inflammation in white matter: Clinical and pathophysiological aspects; Ment Retard Dev Disabil Res Rev; 12(2):141-6), aging (Alvarado C, Alvarez P, Puerto M, Gausseres N, Jimenez L, and De la Fuente M, 2006; Dietary supplementation with antioxidants improves functions and decreases oxidative stress of leukocytes from prematurely aging mice. Nutrition, 22(7-8):767-77), Alzheimer's disease (Di Rosa M, Dell'Ombra N, Zambito A M, Malaguarnera M, Nicoletti F, and Malaguarnera I, 2006; Chitotriosidase and inflammatory mediator levels in Alzheimer's disease and cerebrovascular dementia; Eur J Neurosci, 23(10):2648-56), osteoporosis (Weitzmann M N, and Pacifici R, 2006; Estrogen deficiency and bone loss: an inflammatory tale; Clin Invest. 116(5):1186-94), asthma (Isidori A M, Giannetta E, Pozza C, Bonifacio V, and Isidori A, 2005; Androgens, cardiovascular disease and osteoporosis; J Endocrinol Invest, 28(10 Suppl):73-9), bowel disorders (Zilberman L, Maharshak N, Arbel Y, Rogowski O, Rozenblat M, Shapira I, Berliner S, Arber N, and Dotan I, 2006; Correlated Expression of High-Sensitivity C-Reactive Protein in Relation to Disease Activity in Inflammatory Bowel Disease: Lack of Differences between Crohn's Disease and Ulcerative Colitis; Digestion, 73(4):205-209), psoriasis (Hamminga E A, van der Lely A J, Neumann H A, and Thio H B, 2006; Chronic inflammation in psoriasis and obesity: Implications for therapy; Med Hypotheses, 67(4):768-773) meningitis (Keino H, Goto H, Mori H, Iwasaki T, and Usui M, 2006; Association between severity of inflammation in CNS and development of sunset glow fundus in Vogt-Koyanagi-Harada disease; Am J Opthalmol, 141(6):1140-1142), cystic fibrosis (Clayton A, and Knox A J, 2006; COX-2: a link between airway inflammation and disordered chloride secretion in cystic fibrosis?; Thorax, 61(7):552-553), age related macular degeneration (Seddon J M, George S, Rosner B, and Rifai N, 2005; Progression of age-related macular degeneration: prospective assessment of C-reactive protein, interleukin 6, and other cardiovascular biomarkers; Arch Opthalmol, 123(6):774-82), and cancer (Allgayer H, and Kruis W, 2006; From chronic inflammation to metastasing colon cancer—the endless story of the NSAIDs; Z Gastroenterol, 44(7):611-613). The individual references for the previous sentence are drawn from recent literature to show that studies of this kind are currently one of the most active areas in clinical biomedicine.

The details of these phenomena are being worked out. For example, in neurodegenerative diseases such as Alzheimer's, it has been found that whenever the brain is injured or infected, glial cells in the brain secrete cytokines. Normally, this response shuts down when the injury or infection is over. But in chronic neurodegenerative diseases like Alzheimer's, these glial cells are activated too high or too long or both. The plaques and tangles in patients' brains attract the attention of glial cells, making them secrete even more cytokines to try to repair this damage, and creating chronic inflammation (Ranaivo H R Craft J M Hu W Guo L Wing L K, Van Eldik L J, and Watterson D M, 2006; Glia as a Therapeutic Target: Selective Suppression of Human Amyloid-beta-Induced Upregulation of Brain Proinflammatory Cytokine Production Attenuates Neurodegeneration; J. Neurosci, 26: 662-670).

The role of inflammation in cancer development is under active investigation. It has been discovered that recurrent inflammation and chronic infections actually contribute to a large number of different types of cancers. Tumors arise from chronic inflammation that acts together with chemical carcinogens. A relationship between cancer and inflammation due to chronic infection has been suspected, but not proven, for many years. In a 1986 study, for example, one researcher compared the inflammatory response to a wound healing response, saying tumors were wounds that do not heal. The recent findings establish a role of myeloid cells in inflammation-associated tumor promotion in addition to their role in tumor progression and invasiveness (Greten F R, Eckmann L, Greten T F, Park J M, Li Z W, Egan L J, Kagnoff M F, and Karin M, 2004; IKKbeta links inflammation and tumorigenesis in a mouse model of colitis-associated cancer; Cell, 118(3):285-96).

Modern research is confirming that inflammatory diseases are virtually epidemic and include some of the most devastating afflictions of our times. Over the evolutionary eons, “we developed these important host defenses to let us get to reproductive age,” said Dr. Peter Libby, chief of cardiovascular medicine at Brigham and Women's Hospital in Boston. “Now, the lifespan has almost doubled, and these same [immune responses] contribute to diseases in the end.” Chronic inflammation is so similar in different diseases, Libby said, that when he lectures, he uses many of the same slides, whether he's talking about diseases of the heart, kidneys, joints, lung or other tissues (Foreman J, 2006; Inflammation is Culprit in Many Ailments; On the web at: http://www.myhealthsense.com/F060403_inflamation.html.)

In “The Inflammation Cure,” J. Meggs, MD states that, “Inflammation may turn out to be the elusive Holy Grail of medicine—the single phenomenon that holds the key to sickness and health.” (Meggs W J, and Svec C, 2003; The Inflammation Cure: How to Combat the Hidden Factor Behind Heart Disease, Arthritis, Asthma, Diabetes, & Other Diseases; McGraw-Hill, New York).

These observations and conclusions further relate significantly to the roles of inflammation and ROS free radicals in chronic disease that have been incorporated into an important new theory that has steadily been gaining support within the medical community. The new theory states that the immune reaction generally known as inflammation may be the underlying cause of a wide range of chronic diseases.

As a consequence of current research on inflammation, Time Magazine Newsweek and Scientific American have recently reported that inflammation is emerging as the “Alpha and Omega of disease” . . . that reducing inflammation is the most important thing a person can do to restore their health and prevent disease (Gorman C, Park A, 2005; The Secret Killer. Time Magazine, February 23 issue); (Underwood A, 2005; Quieting a body's defenses; Newsweek Special Edition on the Future of Medicine, Summer issue); (Martindale D., 2005; Reactive Reasoning: Is an inflammation protein the next cholesterol?; Scientific American.com, Mar. 28, 2005).

The familiar manifestations of inflammation should be short lived: swelling, redness, decreased range of motion, heat and pain. However, when the inflammatory response does not shut down properly, inflammation can persist, causing the disruptive manifestations listed in the previous sentence to linger. The resulting discomfort and unnecessary damage to tissues stress the body, prevent proper rest and recovery, and give rise to a host of stress-related disorders (Cohen S, Kessler R C, and Gordon L U, 1995; Strategies for measuring stress in studies of psychiatric and physical disorders. Chapter 1 pp 3-26 in Measuring Stress; Oxford University Press, Oxford, UK) as well as a long list of other problems. Those other problems, known by a variety of disease names, are being recognized as having a common denominator—chronic inflammation.

These problems are particularly significant for the athlete or performer or other person involved in strenuous exertion or physical exercise. The reason for this is that vigorous exertion can increase oxygen intake by a factor of 10 to 20 times. This in turn results in a condition called hyperoxia (elevated oxygen tension in the tissues). Oxygen is a highly reactive and toxic substance (Halliwell B, and Gutteridge J M C, 1999; Oxygen is a toxic gas—an introduction to oxygen toxicity and reactive oxygen species; Chapter 1 in Free Radicals in Biology and Medicine, 3^{rd} edition, Oxford University Press, Oxford, UK), and excess oxygen in the tissues leads to increased intracellular production of oxygen-derived free radicals to levels that can exceed the capacity of the antioxidant defenses that normally remove oxidants. When this happens, free radical damage can overwhelm the restorative processes that normally repair cells and cellular components including DNA. When extreme exertion is coupled with injury, as often occurs in highly competitive sports, the result can be an even larger build-up of free radical damage that can severely inhibit and thereby prolong the recovery process.

The inflammation theory of disease has triggered the search for new anti-inflammatory compounds and other methods for neutralizing excess free radicals. Cortisone was the first steroid drug available. In 1935, researchers at Mayo Clinic, Rochester, Minn., isolated the hormone cortisone from adrenal glands. In 1948, doctors first used the new drug to treat a 28-year-old woman with severe rheumatoid arthritis. Cortisone remarkably relieved her inflamed, swollen joints after just a few days of use. People who normally couldn't climb out of bed or into a bathtub could do so after using the drug. For a long period of time, cortisone injections, also known as cortisol or corticosteroid injections were widely used for reducing pain associated with inflammation. But these drugs do not assist in the healing process. In fact, cortisone has actually been shown to slow healing. This is a central problem in sports medicine. The injured performer gets immediate pain relief from the treatment and is able to continue his or her activity, but this can lead to more serious problems in the longer term.

Because of problems with its side effects, the use of cortisol and related drugs has been largely supplanted with non-steroidal anti-inflammatory compounds (NSAIDS), which are available both by prescription and over-the-counter. As with cortisol, however, experience is showing that prolonged use of NSAIDS can also lead to serious side effects. For example, people who have survived a first heart attack have a higher risk of dying or having a second heart attack if they are taking non-steroidal anti-inflammatory drugs (NSAIDs), including the newer class called cox-2 inhibitors (Salpeter S R, Gregor P, Ormiston T M, Whitlock R, Raina P, Thabane L, and Topol E J, 2006; Meta-analysis: cardiovascular events associated with nonsteroidal anti-inflammatory drugs; Am J Med, 119(7):552-9); (Gislason G H, Jacobsen S, Rasmussen J N, Rasmussen S, Buch P, Friberg J, Schramm T K, Abildstrom S Z, Kober L, Madsen M, and Torp-Pedersen C, 2006; Risk of death or reinfarction associated with the use of selective cyclooxygenase-2 inhibitors and nonselective nonsteroidal antiinflammatory drugs after acute myocardial infarction; Circulation, 113(25):2906-2913).

Many people have turned to vitamins and nutritional supplements thought to have antioxidant and anti-inflammatory properties, but there is debate about the effectiveness of these substances (Vivekananthan D P, Penn M S, Sapp S K, Hsu A, and Topol E J, 2003; Use of antioxidant vitamins for the prevention of cardiovascular disease: meta-analysis of randomised trials; Lancet, 361: 2017-23).

In spite of these difficulties, it is obviously important to develop means to reduce free radical concentrations in tissues, and a variety of chemical methods continue to be disclosed to accomplish this. These methods have the disadvantage that once the antioxidant chemical has reduced a free radical by donating an electron to it, the antioxidant itself can become a free radical. The resulting charge imbalance can be passed in a series of reactions from molecule to molecule, causing further oxidative stress and disrupting metabolism. In addition, the antioxidant, once it has served its purpose, must be metabolized and excreted from the organism, posing an additional work load on the biochemical machinery of the body. Moreover, antioxidants can have deleterious side effects.

The many disadvantages to the prior art related to chemical control of the acute and chronic phases of inflammation are overcome in this invention, which provides direct conductive pathways for natural antioxidant electrons from the earth to rapidly reach sites of inflammation in the body.

It is well established that negative charges (free electrons) are instantly attracted to positive charges (free radicals). (Coulomb's law: The electric force acting on a point charge as a result of the presence of a second point charge ((one positive and one negative)) attract one to the other). (Chemists use the term “electrophile” ((literally electron-lover)) to describe a reagent that attracts electrons. Most electrophiles are positively charged). Connecting the body to the earth automatically enables the conductive tissues of the body to become charged with earth's free electrons. This eliminates the need for free radicals to oxidize healthy tissue to obtain their missing electrons. By readily reducing free radicals with earth's free electrons, oxidation of healthy tissue is inhibited and the immune response winds down properly.

To verify the effects of reducing inflammation in the body with the application of earth's free electrons to the body, several research studies and clinical case studies have been performed. A series of clinical case studies well document the therapeutic effectiveness of reducing chronic inflammation and inflammation-related health disorders by conductively coupling the body with the earth. The rapid reduction of chronic inflammation that is consistently evidenced in the case studies support the concept that nature, throughout evolutionary time, relied upon earth's mobile free electrons as a primary source of antioxidants to reduce residual free radicals in the body. In further considering that the immune system's oxidative response mechanisms developed when humans and animals lived in conductive contact with the earth, the clinical case study results strongly support the concept that the modern practice of wearing synthetic soled shoes and living in environments that insulate the body from the earth is a primary contributor to the modern epidemic of chronic inflammation and related autoimmune diseases.

Current biomedical research confirms that chronic inflammation and inflammation-related diseases are virtually epidemic. They include: high blood pressure, cardiovascular/heart disease, diabetes, multiple sclerosis and other neuromuscular diseases, respiratory disorders, digestive disorders, liver, gall bladder and kidney dysfunction, diseases of the colon, arthritis, chronic fatigue, osteoporosis, hormone imbalances, thyroid dysfunction, Alzheimer's, premature senility/dementia, as well as the continuing rise in cancer.

When the human body is conductively coupled with the earth by means of the present invention, the body naturally conducts and becomes permeated with earth's surface charge of free electrons, i.e. it equalizes with and maintains the natural electrical potential of the earth. In this state earth's free electrons are readily available throughout the body to readily reduce excess free radicals and thereby prevent oxidation of healthy tissue. Current biomedical research (referenced above in Para 16) confirms that free radical oxidation of healthy tissue is the underlying cause of chronic inflammation and autoimmune disease. Clinical case studies show that restoring the earth's natural surface charge of free electrons to the body consistently produces rapid reduction of chronic inflammation.

Accordingly, there is a need for personal body grounding systems which allow earth's mobile free electrons to conductively flow from the earth to the bodies of humans and animals to reduce and prevent chronic inflammation and to treat inflammation-related autoimmune diseases. Such personal body grounding systems should be capable of being used while sleeping, during prolonged periods of sitting, standing and during other activities when the body is residing in an environment that otherwise insulates the body from conductive contact with the earth.

U.S. Pat. No. 6,683,779 discloses a personal grounding system for collecting and removing unnatural electrical charges from a human body. The grounding pad disclosed in the '779 patent comprised between 10% and 20% carbon fibers. A conductor extended across the substrate in conductive contact with the fibers. The grounding pad was configured to make field or conductive contact with the human body. A ground lead was included having a first end conductively coupled to the grounding pad conductor and a second end conductively coupled to a grounded anchor. The grounded anchor was placed directly into the earth. The chair and sleeping pads described in the '779 patent are effective, but it has been discovered that in order for the grounding pad to be in direct conductive contact with the skin of a human body through a covering sheet (in the case of a sleeping pad) or through clothing (in the case of a chair or sitting pad) it is essential for said body to produce perspiration and thereby moisten or hydrate the sheet or clothing, respectively, to render them conductive. Without body perspiration, conductive contact is compromised and only field coupling is established. This slows or prevents the movement of electrons from the earth to the body and thereby reduces the anti-oxidant effects of those electrons.

The '779 patent also allowed for conductive coupling with the body through an adhesive electrode patch or through body bands containing conductive fabrics that could be placed around an ankle, foot or wrist, for example. While these methods are effective, they require a cord be attached to them while being used. This was found to be inconvenient for most users; whereas a conductive footpad or bed sheet requiring only barefoot contact is more convenient for use in most living space and work place environments. The disclosure of the '779 patent is hereby incorporated by reference into this specification.

Experience with use of the previous patent has revealed that re-establishing electrical contact of a human or animal body with the earth produces rapid and extremely valuable anti-inflammatory effects, which therefore favor its application in the treatment of and prevention of numerous acute and chronic inflammatory conditions without interfering with the normal immune responses to tissue injury.

The purpose of the present patent application is to disclose discoveries obtained during the use of the invention described in the '779 patent, and to further disclose improvements and enhancements to the methods of the previous patent based on further research and observation.

### SUMMARY OF THE INVENTION

The invention comprises grounding devices and systems that conduct the earth's mobile free electrons from the earth to the body of an animal or human as would occur in nature if said animal or human were standing barefoot on the earth. Further, the present invention relates to providing an abundant supply of free electrons to the body to reduce or prevent inflammation in the body of an animal or man. The invention generally comprises a body grounding device having a ground lead extending there from that is conductively coupled to a ground system which is connected to an earth ground anchor. The grounding device comprises a grounding pad including a mesh layer substrate that contains between 10% and 20% carbon impregnated mono filament nylon fibers or between 1% and 10% silver coated mono filament nylon fibers. Preferably the mesh layer substrate comprises 95% nylon fibers and 5% silver coated nylon fibers. The fibers are knitted in a cross linking pattern throughout the fabric which allows a conductive connector to be easily placed in the device at any location to maintain conductivity with the fibers. A ground lead is conductively connected at one end to the conductive connector and at another end to an earth ground anchor.

The grounding devices are configured to create a conductive path between the body and the earth. In this regard a grounding device can comprise a conductive fabric covered mattress with a conductive sheet for sleeping on or a half sheet containing conductive fibers that is placed at the foot end of a bed for placing a persons bare feet on during sleep. A grounding device may comprise a conductive floor mat or conductive floor covering for barefoot grounding or with use of conductive footwear to maintain conductive contact between an earthed floor covering or an earthed floor mat and the body when going barefoot is not appropriate. A body grounding device may comprise a chair seat covered with conductive fabrics containing conductive fibers along with clothing that contains conductive fibers to maintain conductive contact between the body and the conductive fibers in a chair seat covering. A grounding device may also comprise a desk pad or computer mouse pad covered with fabrics containing conductive fibers for conductive hand or wrist contact. A grounding device may also comprise an adhesive electrode patch or conductive fabric tape or body wrap for use in grounding localized areas of inflammation in the body. A disposable one-time use grounding device for clinical applications may comprise a non-woven material containing conductive carbon fiber.

Preferably, the ground anchor is placed directly in the earth with a ground wire leading to a living or workplace environment that terminates in a wall type outlet, i.e., an electrical outlet ground port. In modern homes the earth ground wire of a working electrical ground system may be used. A removable ground lead wire is connected to the connector of a grounding device and connected to the wall outlet ground system. When existing electrical ground wire systems are used it is preferable to have a test circuit that plugs into the outlet and tests for proper outlet wiring which confirms with a light that a working earth ground is connected to the ground port. This test circuit is to remain in the electrical outlet to continuously confirm that a working ground exists.

The device is further configured to provide a system interface device providing an electrical contact terminal or multiple terminals to connect grounding devices to the earth ground system. For safety the interface ground port outlets may contain an inline meter, fuse or current limiting resistor. Further, a noise reduction component may be placed inline on the ground port side within the earth ground interface device to reduce electrical noise that may exist on the ground system.

Other features and advantages of the present invention will become apparent from the following more detailed descriptions, taken in conjunction with the accompanying drawings which illustrate, by way of example, the principles of the invention.

### BRIEF DESCRIPTION OF THE DRAWINGS

The accompanying drawings illustrate the invention. In such drawings:

FIG. 1 is a schematic view of a barefoot grounding system embodying the present invention comprising a conductive floor mat, for positioning on a floor and directly connected to an earth ground anchor.

FIG. 2 is a view of the conductive fibers running throughout the conductive layer of the floor mat.

FIG. 3 is an enlarged view of a conductive snap connector in contact with the conductive fibers.

FIG. 4 is a cross-sectional view taken generally along the line 4-4 of FIG. 3, illustrating he conductive snap connector extending through the conductive fibers and mat.

FIG. 5 is a perspective view of a barefoot grounding system embodying the present invention comprising a conductive wide area floor covering, for positioning on a floor and directly connected to an earth ground anchor.

FIG. 6 is a perspective bottom view of footwear in accordance with present invention comprising a conductive bottom layer and a conductive top layer that are conductively coupled together with a conductive insert to provide conductivity between a barefoot and a conductive floor covering.

FIG. 7 is a top perspective view of the footwear of FIG. 6.

FIG. 8 is a cross-sectional view taken generally along line 8-8 of FIG. 7, illustrating a conductive insert extending between the conductive top layer and the conductive bottom layer, in accordance with the present invention.

FIG. 9 is a perspective view of use of FIG. 5 footwear to make conductive contact with a wide area conductive floor covering that is connected an earth ground anchor.

FIG. 10 is a perspective view of a conductive shoe sole insert, for use in conventional leather soled shoes or conventional conductive soled shoes, with a conductive fabric top layer and a conductive fabric bottom layer, conductively coupled together with a conductive insert.

FIG. 11 is a perspective bottom view of FIG. 9 conductive fiber fabric bottom and conductive insert.

FIG. 12 is a cross-sectional view taken generally along line 12-12 of FIG. 11, showing a conductive top layer, a dielectric polymer foam layer in between and a conductive bottom layer with a conductive insert conductively connecting the top and bottom layers.

FIG. 13 is a cross-sectional view of a conventional leather soled or conventional conductive soled shoe with the conductive insert of FIG. 10 included therein.

FIG. 14 is a partially fragmented view of a chair seat covered with fabric containing conductive fibers that are conductively connected with a snap connector to ground lead that is connected to a ground system connected to an earth ground anchor.

FIG. 15 is a perspective view of clothing containing conductive fibers.

FIG. 16 is a perspective view of a personal grounding system embodying the present invention via a person wearing FIG. 15 conductive clothing while sitting on FIG. 14 chair with conductive fibers conductively connected via a ground lead to a ground system that is connected an earth ground anchor.

FIG. 17 is a schematic view of a personal grounding system comprised of a mattress with the bottom half covered in fabric containing conductive fibers that are conductively connected with a metal snap to a ground lead that is connected to a ground system that is connected to an earth ground anchor.

FIG. 18 is a perspective view of a fitted bottom sheet containing conductive fibers.

FIG. 19 is a perspective view of FIG. 17 mattress covered with FIG. 18 sheet with the conductive fibers of the sheet making conductive contact with the conductive fibers contained in the bottom portion of the mattress which is connected via a ground lead to a ground system that is connected to an earth ground anchor.

FIG. 20 is a perspective view of a half sheet containing conductive fibers that is placed at the foot end of bed and conductively connected via a ground lead to a ground system that is connected an earth ground anchor.

FIG. 21 shows a half sheet containing conductive fiber in the sheeting fabric with a metal snap connector placed therein making conductive contact with conductive fibers.

FIG. 22 is a side view of snap making contact with conductive fibers.

FIG. 23 is a perspective view of a personal grounding system embodying the present invention comprising a sleeping bag containing conductive fibers connected via a conductive snap connector to a ground lead that is connected to an earth ground anchor.

FIG. 24 is a schematic view of a fabric strip containing conductive fibers with a metal snap connector fastened at one end.

FIG. 25 is a side view of FIG. 24 showing snap connector securely attached to conductive fabric strip.

FIG. 26 is the reverse side if FIG. 24 showing an adhesive layer with a removable protective covering.

FIG. 27 shows adhesive protecting covering being removed.

FIG. 28 illustrating FIG. 24 conductive fabric strip being applied to the body with a ground wire extending there from and connecting to an earth ground anchor.

FIG. 29 is a perspective view of a conventional pet bed covered with fabric containing conductive fibers connected via a conductive snap connector to a ground lead attached to an earth ground anchor.

FIG. 30 is a perspective view of a conventional desk mat or computer mouse pad covered with fabric containing conductive fibers connected via a conductive snap connector to a ground lead attached to an earth ground anchor.

### DETAILED DESCRIPTION OF THE PREFERRED EMBODIMENTS

The present invention relates to an improved personal body grounding system that can restore and maintain the natural electrical balance of the human body. More particularly, the present invention relates to personal grounding devices and systems that conduct the earth's mobile free electrons, from the earth, to the body of an animal or human, similar to that which would occur in nature if the animal or human were standing barefoot on the earth, and thus provide an abundant supply of free electrons to the body to reduce and prevent metabolic and residual immune system-produced free radicals from oxidizing normal tissue and promoting chronic inflammation in the body. The system also couples the body to the oscillating electrical field of the earth that serves to synchronize physiological rhythms.

Sufficient conductive contact between the body and the surface of the earth, and its beneficial consequences, can be achieved most naturally and preferentially through the bare feet. This contact is preferably established via direct contact between the feet and a conductive footpad or bed sheet. In the preferred an embodiment of the present invention, conductivity is established with fabrics containing silver fibers. Silver provides improved conductive coupling with the feet and also gives the invention advantageous anti-microbial, anti-bacterial and anti-fungal properties. This disclosure provides advantages and enhancements over the '779 patent, that disclosed grounding comprising a sitting or sleeping pad and including a mesh layer substrate comprised of a plurality of electrically conductive carbon fibers.

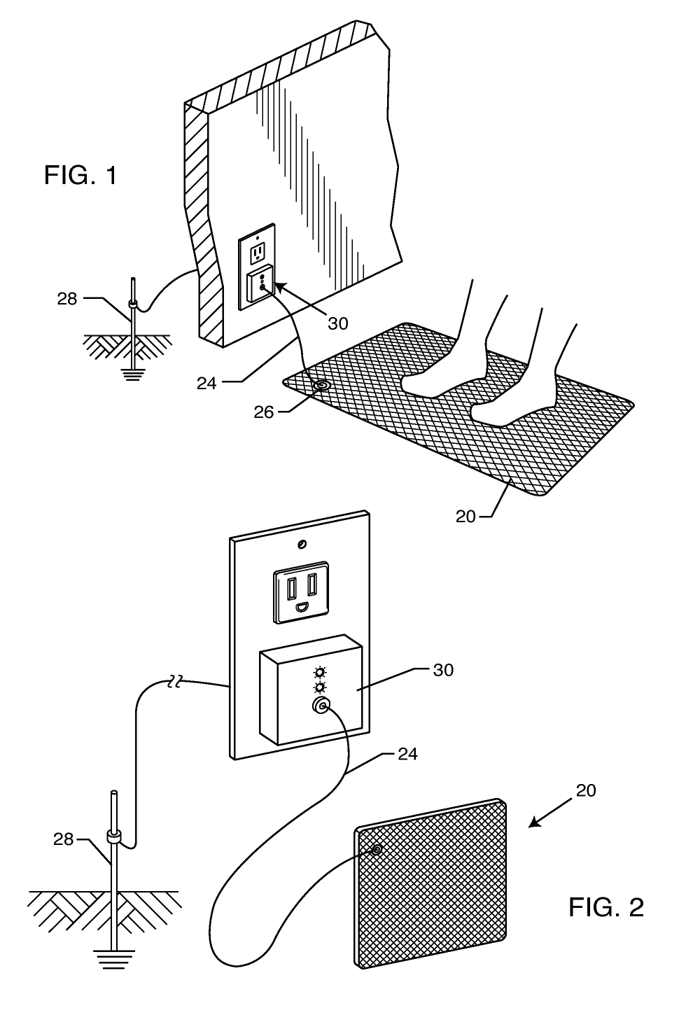

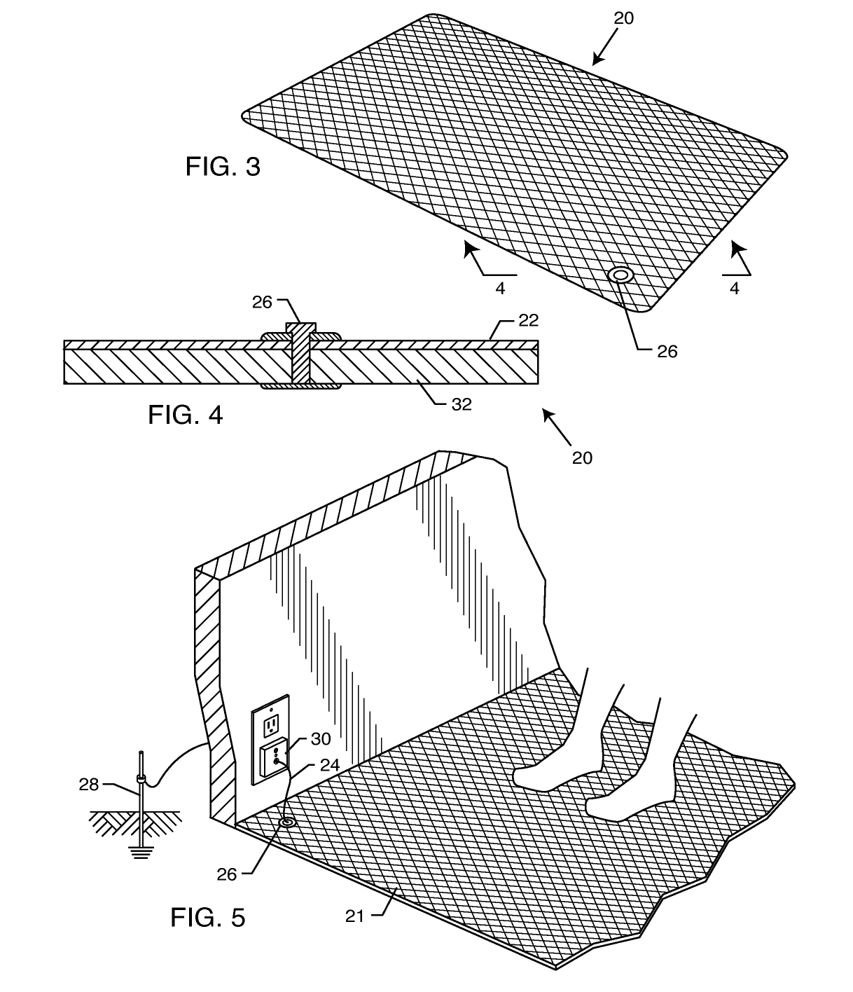

With reference to FIG. 1, the system includes a grounding pad 20 in the form of a floor mat for placement under a computer desk or other work area. The pad 20 is sufficiently large enough for a person to maintain barefoot contact with the pad no matter how a person positions their feet when sitting at a desk. The pad 20 includes a mesh layer substrate 22 illustrated in FIG. 4 which is comprised of a plurality of silver coated nylon fibers. Typically the substrate 22 comprises 95% nylon fibers and 5% silver coated nylon fibers knitted into a conductive grid pattern. Although 5% silver fiber content is preferred, the silver fiber content may range between 1% and 10% silver fibers.

The use of silver in this application provides further advantages. Silver is the most electrically conductive metal. Its thermal properties are advantageous in temperature regulation because silver is thermally the most conductive and reflective element. The use of silver in a bed sheet effectively dissipates heat and keeps the users cooler in hot climates and warmer in cold climates. Silver is recognized in modern medicine as the most effective antimicrobial agent known. It outperforms all natural and synthetic products with a 99.9% kill rate of over 800 micro-organisms in analyses conducted in accordance with the relevant standards and testing procedures. Silver fibers kill many bacteria, including odor-causing forms, within one hour of exposure. Silver is also effective at killing “tough” microbes that currently have no other treatment options, such as Staphyloccous aureus. Silver bandages are currently class 1 and class 2 FDA approved medical devices in the United States, and silver is also a registered pesticide with the Environmental Protection Agency (EPA), and there are no known side effects from use of silvered products. No microbe has ever been proven to develop a resistance to silver treatment. Silver as used in this invention may also assist in the treatment of bedsores by killing bacteria which in turn promotes healing.

A ground lead 24 is connected at a first end thereof to the conductive snap connector 26 which is in contact with the silver fibers of the conductive mesh grid of the pad 20 as illustrated in FIG. 4. The ground lead 24 is comprised of a conductive material, such as a copper wire. The ground lead is of sufficient length to extend from the grounding pad 20 to an earth ground anchor 28. Alternatively, the ground lead 24 is of sufficient length to attach to an electrical outlet ground port 30 as illustrated in FIG. 2.

As described in the '779 patent, one end of the ground lead can include a connector such as a banana clip which can be removably attached to a wall plate. Although the connector can comprise a plug, such as a banana clip, for insertion into an outlet-like aperture of a wall plate, the connector may also comprise a snap-fit connector of the circular or ring variety which can be snapped onto a mating member of the wall plate so that children cannot accidentally insert the connector into an electrical socket. Alternatively or additionally a snap-fit connector of the circular or ring variety can be snapped onto a mating member coupled to the grounding pad.

A meter or fuse or current limiting resistor may be associated with the system, and typically installed within the ground lead 24 or electrical outlet ground port 30, as illustrated in FIG. 1. The fuse or current limiting resistor is intended to prevent potential harm from an electrical event.

With reference again to FIG. 2 which illustrates a schematic view of a ground system interface device 30 related to the present invention comprising a box containing electrical contact terminals that are connected to a test circuit residing in the device 30 that tests the wiring configuration of the outlet and confirms that an earth ground wire is connected to the third prong electrical outlet port. If the outlet wiring is correct and a ground wire exists a green light will indicate that the electrical outlet earth ground is in working order. This device will also contain banana jack type ground ports for connecting ground leads from personal grounding devices to the earth ground system as illustrated. Each ground port outlet of the device will contain an inline current limiting resistor, such as a 1 megohm resistor, which meets current established standards to protect a grounded person against harm from an electrical event. Further, a noise reduction component may be placed inline on the ground port side within the interface ground device to reduce electrical noise that may exist on the earth ground system. Finally, those familiar with electrical wiring systems will be aware that appropriate modifications of the ground interface device will be required in different countries that utilize different types of electrical outlets and wiring configurations.

With reference now to FIG. 5 the system comprises a wide area floor covering 21 large enough in size for a person to maintain barefoot contact with the covering when walking or standing in various areas of an average size room. The grounding pad 21 is similar to the grounding pad 20 described above in that it includes a plurality of conductive fibers to form a conductive grid as described above. The grounding pad 21 includes a foam base 32 for cushioning purposes. Similar to that described above the system includes a ground lead 24 interconnected to the conductive connector 26 of the pad and an earth ground system.

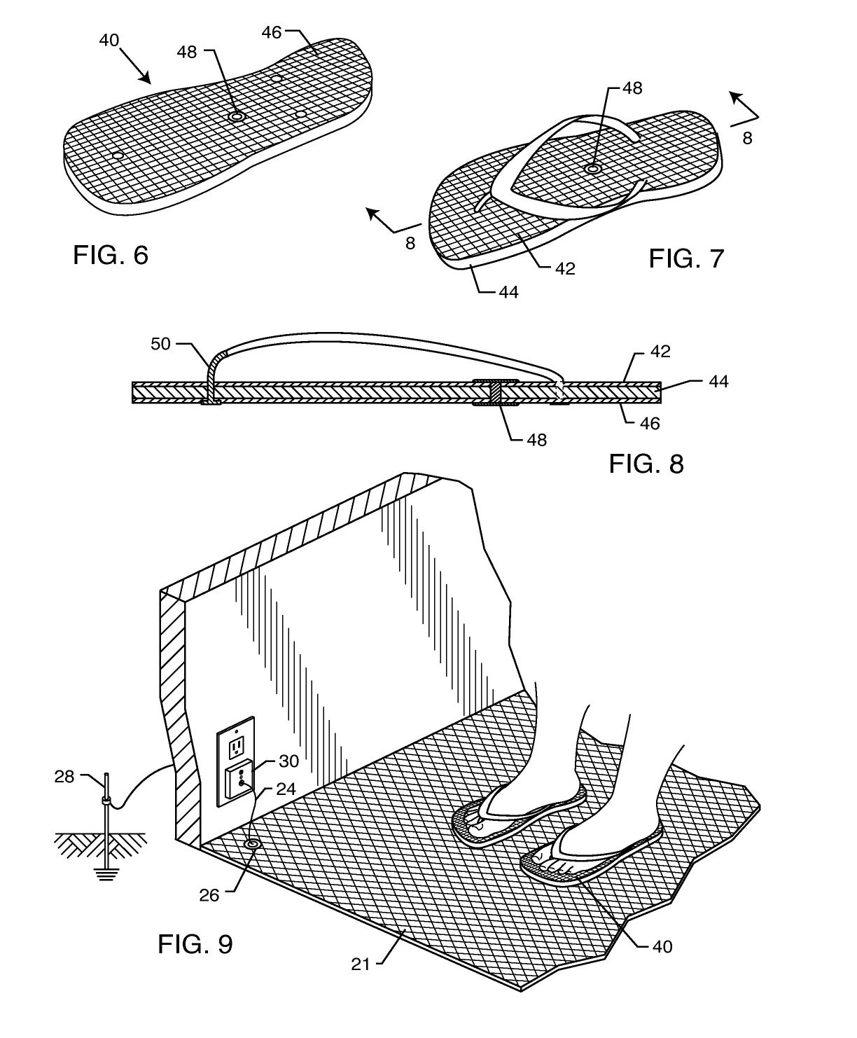

With reference now to FIGS. 6-9, the system comprises conductive footwear 40 to be worn as illustrated in FIG. 9 when a person needs to be grounded for extended periods for health reasons but it is not permissible to go barefoot in a particular work or living environment for cultural or sanitary or safety reasons. The conductive footwear 40 as shown in FIG. 8 is comprised of a conductive top layer 32, a center layer of foam 44, and a conductive bottom layer 46 with a conductive rivet 48 conductively connecting the top and bottom conductive layers 42 and 46. This provides a conductive path between the bottom of a person's bare foot and the conductive floor mat 21 that is connected to an earth ground anchor 28. The conductive top and bottom portions of the footwear 40 contain 1%-10%, preferably 5%, silver fibers in a mesh grid as outlined above that is bonded to the foam center portion of the foot pad. FIG. 8 illustrates use of a thong 50 type apparatus for holding the conductive foot pad on the foot. The thong may be composed of plastic containing carbon to create conductivity between a person's bare feet and a conductive floor covering as illustrated FIG. 9.

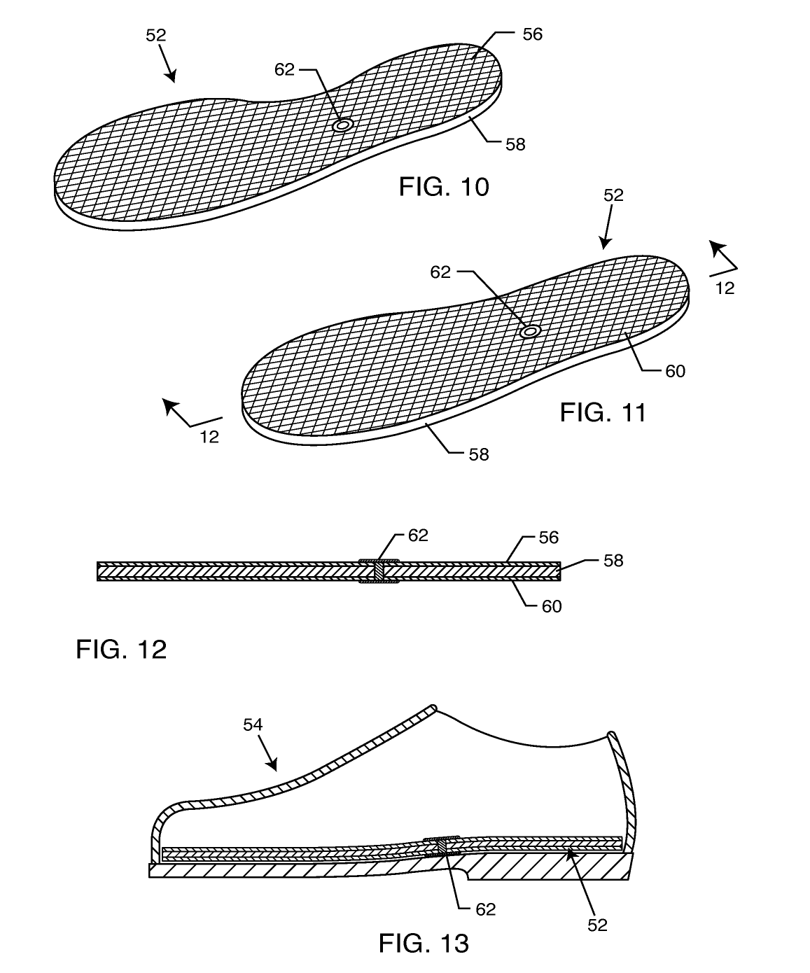

With reference now to FIGS. 10-13, the system comprises a conductive shoe sole insert 52 to replace nonconductive inserts in conventional leather soled shoes or conventional conductive soled shoes 54 to make conductive contact between a persons bare feet and a conductive floor covering 21 as illustrated in FIG. 9 or a conductive floor pad 20 as illustrated in FIG. 1. As also illustrated in FIG. 12, the bottom conductive fabric layer 60 of the insert contains 1%-10%, preferably 5%, silver fibers in a mesh fabric grid that is bonded to the foam core 58 of the insert. FIG. 12 shows a conductive rivet 62 making contact with both the top and bottom conductive fabrics 56 and 60. FIG. 13 shows the conductive insert placed in a conventional leather soled shoe 54 which would also resemble placement in a conventional conductive soled shoe. It is widely known that leather soled shoes, after worn for only a few days, becomes saturated with body salts and perspiration that make leather soles conductive. This is why electricians' do not wear leather soled shoes when handing or working around live electrical wires.

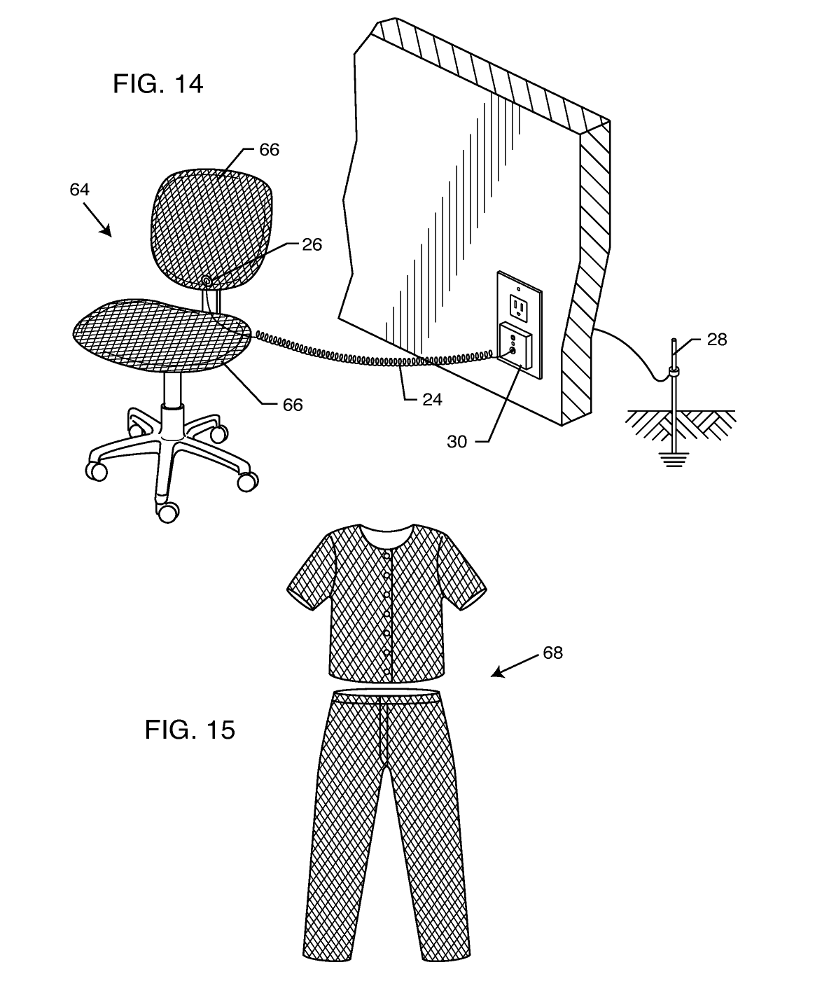

With reference now to FIG. 14 the system illustrated comprises a chair seat 64 covered with fabric 66 that contains 1%-10%, preferably 5%, silver coated nylon fibers woven in a grid with a conductive snap 26 connector attached to the fabric for connecting first end of a ground lead 24 to the conductive fibers of the fabric 66 and the second end connecting to ground system that is connected to an earth ground anchor 28. Conductive contact between the body and the conductive seat pad is made via hydration of clothing from normal body perspiration.

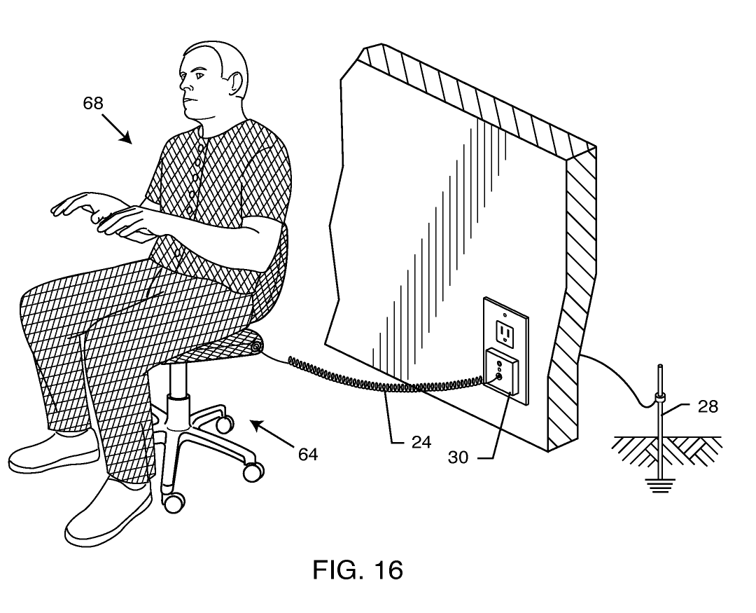

With reference now to FIG. 15 the system comprises clothing 68 made from fabric that contains conductive silver coated nylon fibers. The conductive silver fibers are woven in a pattern throughout the fabric to create a conductive path between the body and a conductive chair seat as illustrated in FIG. 16 showing a person sitting on a conductive chair seat that is connected to a ground lead that is connected to an earth ground system. Although 5% silver fiber content is preferred for clothing fabrics, the silver fiber content may range between 1% and 10% silver fibers.

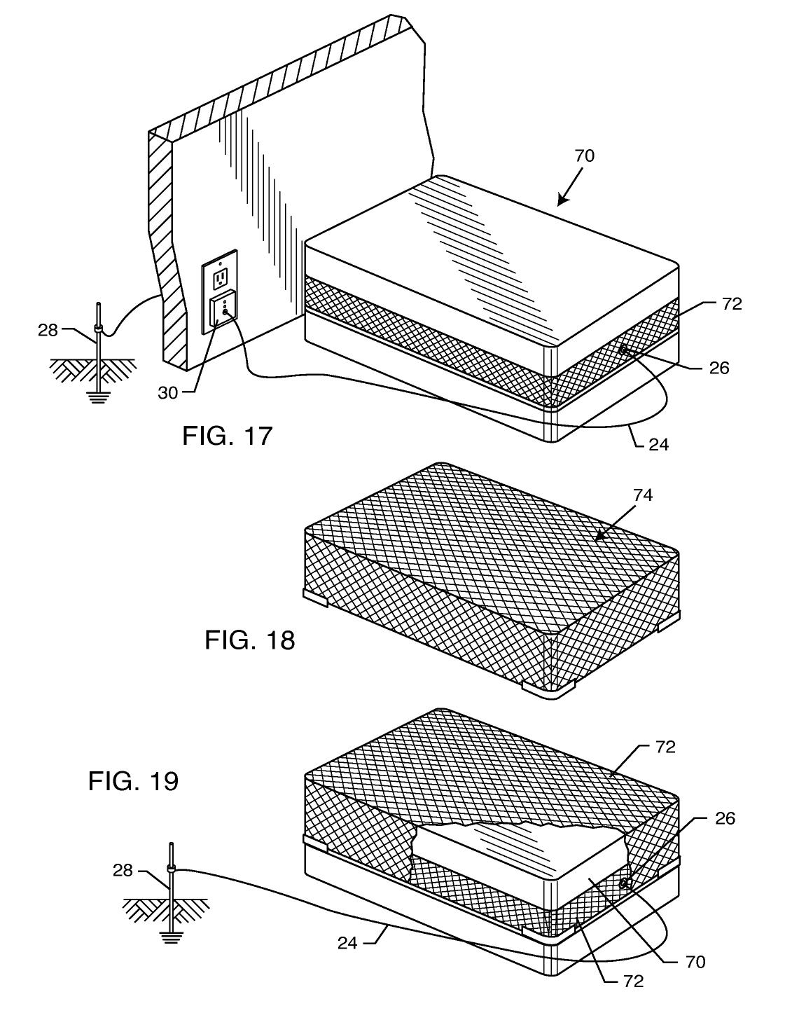

With reference now to FIG. 17 the system illustrated comprises a mattress 70 with ticking on the lower portion (foot end) of the mattress 72 that contains 1%-10%, preferably 5%, conductive silver fibers woven into a conductive grid pattern throughout the ticking with a conductive snap 26 placed through the ticking to conductively connect the first end of a ground lead 24 that is connected to an earth ground system 28 and 30. Mattress 70 illustrated in FIG. 17 is to be covered with sheet 74 illustrated in FIG. 18, which is comprised of 95% cotton fibers and 5% silver coated nylon fibers with the silver fibers woven in a conductive grid pattern throughout the sheet. Although 5% silver fiber content is preferred, the silver fiber content may range from 1% to 10%. When the sheet 74 is placed over the mattress 70 as illustrated in FIG. 19, the bottom portion of the sheet 74 makes conductive contact with the conductive fibers of the bottom portion 72 of the mattress which is connected via a ground lead to an earth ground system as illustrated in FIGS. 17 and 19.

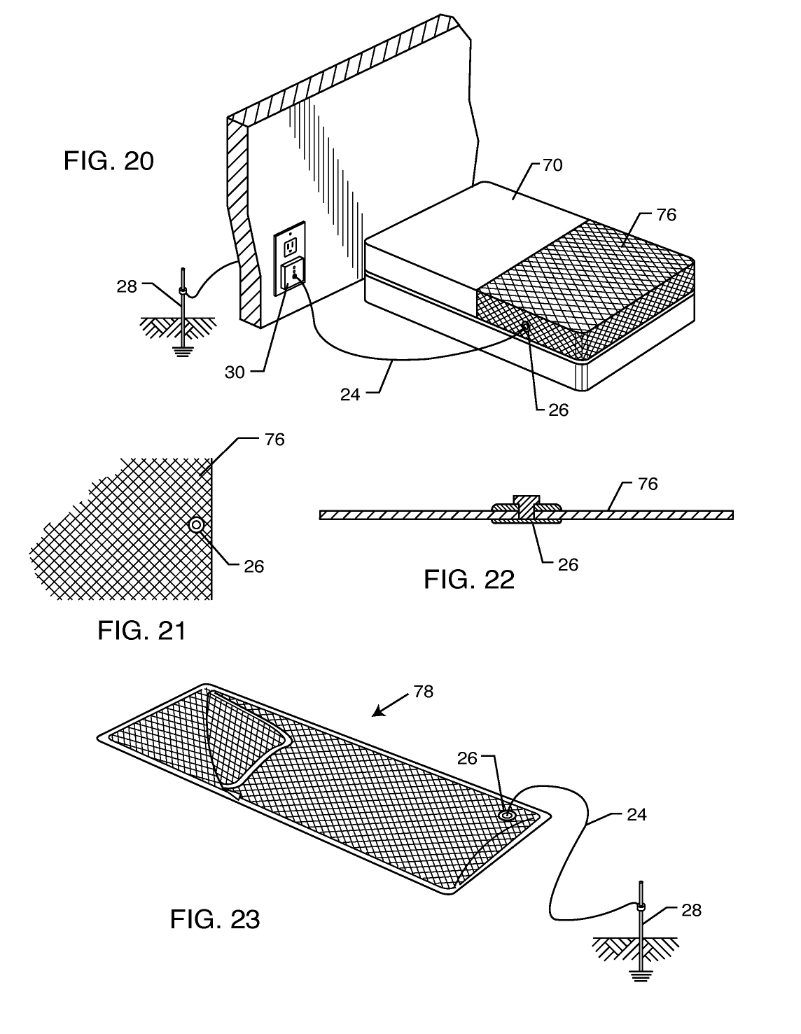

With reference now to FIG. 20, a system comprised of a half sheet 76 containing conductive silver coated fibers, as described above in reference to sheets illustrated in FIG. 18, which is placed at the foot end of the bed 70 on top of the existing bottom fitted sheet and conductively connected to the first end of a ground lead 24 that connects at the other end to an earth ground system 28. FIG. 21 shows the conductive snap connector placed in the half sheet 76 for connecting a conductive ground lead. FIG. 22 is an enlarged view of the conductive snap connector of the half sheet in conductive contact with the silver fibers that are woven in a cross linking pattern throughout the sheet. The sheet is sufficiently large enough that a person's bare feet will remain on the sheet during sleep, no matter what position the person's feet are in during sleep.

With reference now to FIG. 23 illustrating a ground system comprised of a sleeping bag 78 made with the sheeting material described in FIG. 18 above that contains a conductive snap connector 26 that is connected to the first end of a ground lead 24 with the other end connected to an earth ground anchor 28.

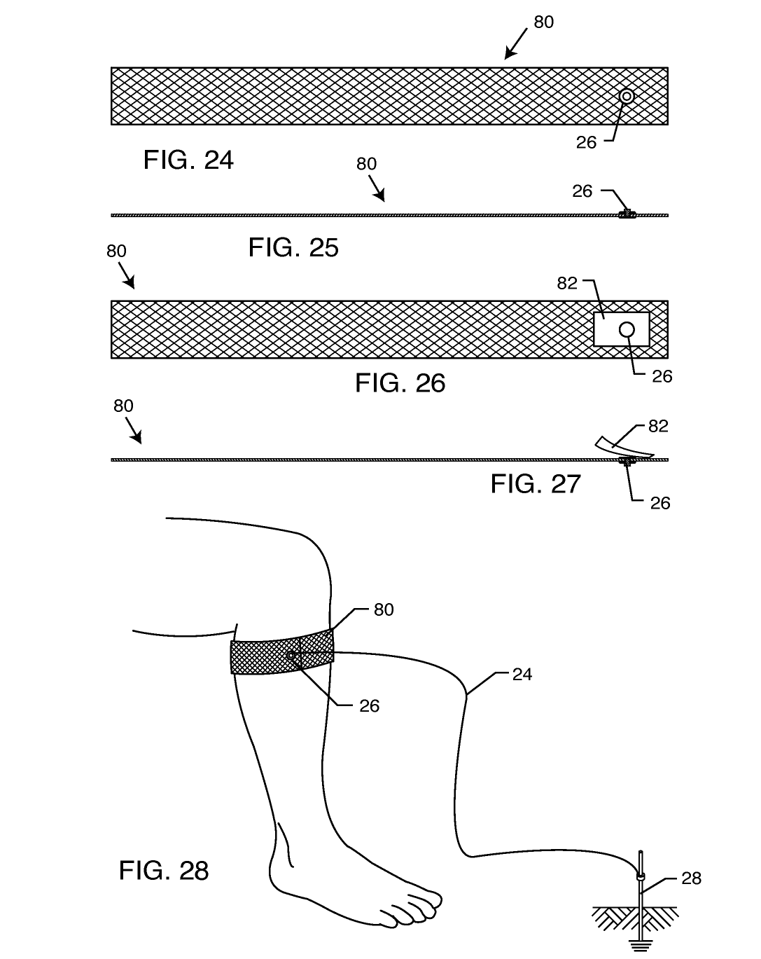

With reference now to FIG. 24 a system comprised of a fabric tape strip 80 containing conductive silver coated nylon fibers woven in a conductive grid throughout the fabric 80 with a conductive snap connector 26 that makes conductive contact with the silver fiber at one end of the fabric strip. FIG. 25 illustrates a side view of fabric tape strip snap connector 26. FIG. 26 illustrates an adhesive layer 82 on one side of the strip with a protective covering and FIG. 27 illustrates removal of the protective covering prior to application of the tape strip to the body. FIG. 28 illustrates use of the conductive strip being used to ground an area of the body with a ground lead 24 attached to the conductive snap connector 26 with the first end of ground lead and the other end connected to an earth ground anchor 28. The length and width of the conductive fabric tape may vary, such as from 12 to 240 inches in length and from 2 or more inches in width.

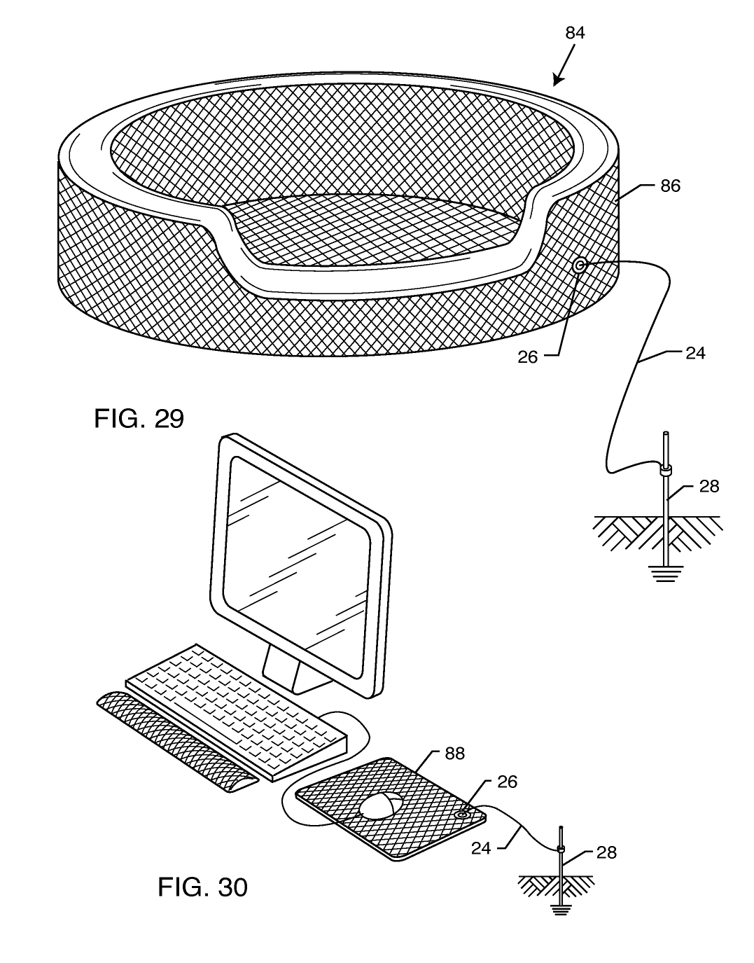

With reference now to FIG. 29 a system comprised of an animal bed 84 covered with fabric 86 that contains 5% conductive silver coated nylon fibers that are woven in a conductive grid pattern throughout the fabric with a snap connector 26 attached to the fabric in contact with the silver fibers that is conductively connected to the first end of a round lead with the other end connected to an earth ground anchor 28. The size of conductive animal beds will vary similar to beds sold in pet stores for varying sizes of animals.

With reference now to FIG. 30 a system comprised of a desk or computer mouse pad or wrist pad covered with fabric 88 that contains 1%-10%, preferably 5%, conductive silver coated nylon fibers that are woven in a conductive grid pattern throughout the fabric with a snap connector 26 attached to the pad 88 in contact with the silver fibers that is conductively connected to the first end of a ground lead 24 with the other end connected to an earth ground anchor 28. The size of the conductive desk pad 88 may vary similar to desk pads and computer mouse pads normally used in office and work space environments.

Although several embodiments of the invention have been described in detail for purposes of illustration, various modifications may be made without departing from the spirit and scope of the invention. Accordingly, the invention is not to be limited, except as by the appended claims.

What is claimed is:

1. A personal body grounding system, comprising:
a grounding pad including a mesh layer substrate containing silver coated nylon fibers;
a conductive connector on the grounding pad conductively coupled to the silver coated nylon fibers;
a grounding lead conductively connected at a first end to the conductive connector and at a second end to an earth ground anchor.

2. The personal body grounding system of claim 1, wherein the mesh layer substrate is comprised of 1% to 10% of silver coated nylon fibers.

3. The personal body grounding system of claim 2, wherein the mesh layer substrate is comprised of 95% nylon fibers and 5% silver coated nylon fibers.

4. The personal body grounding system of claim 1, wherein the grounding lead is connected to the earth ground anchor through an electrical outlet ground port.

5. The personal body grounding system of claim 1, further comprising a meter, fuse or current limiting resistor between the grounding lead and the earth ground anchor.

6. The personal body grounding system of claim 1, further comprising a system interface device between the grounding lead and the earth ground anchor.

7. The personal body grounding system of claim 6, wherein the system interface device includes multiple electrical contact terminals for connecting multiple grounding pads thereto.

8. The personal body grounding system of claim 6, wherein the system interface device includes a test circuit for testing continuity to ground.

9. The personal body grounding system of claim 6, wherein the system interface device includes an inline meter, fuse or current limiting resistor between the ground lead and the earth ground anchor.

10. The personal body grounding system of claim 1, wherein the grounding pad comprises a floor covering, a floor mat, conductive footwear, a conductive chair seat, a conductive mattress, a conductive bed sheet, a conductive sleeping bag, a conductive fabric tape strip, a conductive animal bed or a computer mouse pad.

11. The personal body grounding system of claim 10, wherein the conductive footwear is a thong type sandal or shoe sole insert having conductive top and bottom layers, a center foam layer and a conductive rivet therethrough conductively connecting the top and bottom layers.

12. A personal body grounding system, comprising:
a grounding pad including a mesh layer substrate containing 1% to 10% of silver coated nylon fibers;
a conductive connector on the grounding pad conductively coupled to the silver coated nylon fibers;
a grounding lead conductively connected at a first end to the conductive connector and at a second end to an earth ground anchor; and
a system interface device between the grounding lead and the earth ground anchor.

13. The personal body grounding system of claim 12, wherein the mesh layer substrate is comprised of 95% nylon fibers and 5% silver coated nylon fibers.

14. The personal body grounding system of claim 12, wherein the grounding lead is connected to the earth ground anchor through an electrical outlet ground port.

15. The personal body grounding system of claim 12, further comprising a meter, fuse or current limiting resistor between the grounding lead and the earth ground anchor.

16. The personal body grounding system of claim 12, wherein the system interface device includes multiple electrical contact terminals for connecting multiple grounding pads thereto.

17. The personal body grounding system of claim 12, wherein the system interface device includes a test circuit for testing continuity to ground.

18. The personal body grounding system of claim 12, wherein the system interface device includes an inline meter, fuse or current limiting resistor between the ground lead and the earth ground anchor.

19. The personal body grounding system of claim 12, wherein the grounding pad comprises a floor covering, a floor mat, conductive footwear, a conductive chair seat, a conductive mattress, a conductive bed sheet, a conductive sleeping bag, a conductive fabric tape strip or a conductive animal bed.

20. The personal body grounding system of claim 19, wherein the conductive footwear is a thong type sandal or shoe sole insert having conductive top and bottom layers, a center foam layer and a conductive rivet therethrough conductively connecting the top and bottom layers.
